(19) United States
(12) Patent Application Publication
(10) Pub. No.: US 2019/0365956 A1
(43) Pub. Date: Dec. 5, 2019

(54) METHOD AND APPARATUS FOR USING ELASTOMERIC MATERIALS IN SURGICAL APPLICATIONS

(71) Applicant: BÖNWRX LTD., Lansing, MI (US)

(72) Inventors: Ralph CARMICHAEL, Lansing, MI (US); Clayton Cabot CARMICHAEL, Lansing, MI (US); Erengul Rose CARMICHAEL, Lansing, MI (US)

(73) Assignee: BONWRx, LTD., Lansing, MI (US)

(21) Appl. No.: 16/480,244

(22) PCT Filed: Jan. 23, 2018
(86) PCT No.: PCT/US2018/014846

§ 371 Date: Jul. 23, 2019

Related U.S. Application Data
(60) Provisional application No. 62/449,337, filed on Jan. 23, 2017.

(51) Int. Cl. A61L 27/54; A61F 2/30

(52) U.S. Cl. CPC A61L 27/54; A61L 27/18; A61F 2/30

(57) ABSTRACT

A method of preparing an implantable spacer device utilizing an elastomeric material containing antibiotic(s), radiation-emitting seeds, and/or other bio-active substance to treat diseased, infected and/or injured bony structures, soft tissue, or neural structures. The elastomeric material may include silicone elastomers.

### CROSS-REFERENCE TO RELATED APPLICATIONS

This application claims the benefit of U.S. Provisional Application No. 62/449,337, filed on Jan. 23, 2017. The entire disclosure of the above application is incorporated herein by reference.

### INTRODUCTION

This section provides background information that is not necessarily in the prior art. The use of cured, pre-formed silicone implants in the human body has been a very successful and beneficial therapy for many issues. Recently, a novel in situ curing silicone system has been created which allows the silicone to be implanted as a flowable or malleable paste. The implant then solidifies and cures in place, creating a solid, non-resorbable implant that adheres and is formed precisely to the surrounding tissue. The ability of a surgeon or clinician to form the implant directly to the affected or intended area may also create several novel systems and applications which have added benefit to therapies and implants currently available.

Alternatively, a silicone elastomer may be formed into a particular shape using a mold or other shaping method and allowed to cure outside of the body, for example, in a laboratory or an operating room to create a solid, molded implant prior to use in a patient or other application. Also, pre-made solid implants may be used in the present invention. The pre-made implants may be custom-made and/or off-the-shelf implants of various shapes, sizes and/or applications.

The pre-made implants may be ordered for a particular application and/or acquired from other entities and used in the present invention.

### SUMMARY

The present invention relates to a method of preparing or creating an implantable spacer device utilizing an elastomeric material (for example, a polymer) which contains antibiotic(s), radiation-emitting seeds, and/or other bio-active substance to treat diseased, infected and/or injured bony structures, soft tissue, or neural structures in a living creature, in particular, a human being. However, applications for treating animals are also provided. This invention also relates to a composition suitable for use in such a method, its preparation and use. The elastomeric polymers may include, but are not limited to, silicone elastomers. As used herein, the terms elastomeric polymer, silicone elastomer, and polymer may be used interchangeably.

Further, a combination therapy using a bio-active substance or a non-bio-active substance such as internal radioactive “seeds” and an implant of silicone elastomer, whether the elastomer is cured in situ or outside of the body, may be created with one or more bio-active substances from the following list, in any combination: any pharmaceutical, drug, medicine, medicament, etc. approved for human or animal use, as applicable, anti-biotics, chemo-therapy agents, bone morphogenic proteins, anti-tumor drugs, anti-inflammatory agents, steroid hormones (such as cortisone), hormones, pain medications, anti-viral drugs, and interstitial radiation seed. Other drugs, medicaments, solutions, chemicals, medicines and the like may also be used and are considered to be within the scope of the present invention. Those having ordinary skill in the art may recognize other additives and/or substances that may be used with the present invention. The combination implant and bio-active and radiation seed implant substance therapy proposed may also benefit with the use of generic drugs. The use of any bio-active, radiation or pharmaceutical substance with the in situ curing silicone system may create an implant which may stay in place, and may elute, or release over time, the medicaments or bio-active or radiation substances incorporated or mixed with the pre-cured silicone. The addition of an opacity agent, such as barium sulfate, silver, zirconium, platinum, and/or any other agents may also allow the clinician to easily monitor the location of each implant.

The ability of the in situ curing silicone system to readily conform to the surrounding tissue and geometry gives the implant the ability to “custom fit” each patient and implantation site. An important feature of this conformity to the detailed structure in the implant location may be the increased surface area which may be in contact with the tissue at the location. FIG. 5 shows one example of the improvement of surface area contact with bony structures as compared to Polymethylmethacrylate (PMMA).

The ability of the in situ curing silicone system to provide increased surface area contact with the surrounding tissue is an important benefit in the chemo-therapy or anti-tumor agents, where the material which may be eluting from the silicone implant may be an aggressive bio-active or radiation-emitting material. By increasing the direct contact at the desired area and increasing the surface area contact, it may be feasible that a lower concentration, or strength, of the bio-active or radiation-emitting substance may be utilized, thus potentially reducing morbidity or undesired side effects from the treatment. One such application may be the removal of a tumor, followed by filling the void left by the tumor with the in situ curing silicone system in combination with any bio-active substance, anti-tumor, chemotherapy, pain or antibiotic drug, radiation emitting substance, or any combination thereof. The cured implant may not only fill the void for potential cosmetic, such as reconstructive and/or cosmetic plastic surgery, but may provide biomechanical benefit and/or assist in local inflammation, pain, biomechanical deficit and/or tumor treatment.

Another example of a beneficial combination of the in situ curing silicone system with bio-active substances is Total Joint Arthroplasty (TJA). A detailed discussion and explanation of the benefits of the in situ curing silicone system in tandem with bio-active substances may be explored using TJA as a specific example. TJA is a surgical procedure for the treatment of severe arthritis and other disorders in which the normal articulating surfaces of a joint are replaced by metal and plastic prostheses. The operation most commonly involves replacement of the hip joint, although the knee, shoulder, and other joints may also be replaced. This disclosure shall not be limited to the hip joint even though the figures primarily illustrate the hip joint. The majority of these replacement procedures result in an acceptable functioning joint for the patient, with any infection issues being resolved through the use of standard, systemic antibiotics. However, for a small population of these implant recipients, the standard attempts to treat the infection are unsuccessful, and secondary intervention may be required.

Although the risk of infection after arthroplasty has decreased over the last three decades with the use of prophylactic antibiotics, laminar airflow operating rooms and whole-body exhaust suits, deep infection after arthroplasty remains a serious complication. Significant morbidity to the patient and the cost to the health care system remain. During this period of time, diagnostic techniques also have improved. Treatment options now include: suppressive antibiotics, irrigation and debridement with retention of components, one-stage re-implantation, two-stage re-implantation, and salvage procedures. Based on the medical literature, the successful eradication of a total joint replacement infection with a two-stage re-implantation protocol is over 90% while the success rate with a one-stage protocol is approximately 80%. Surgical protocol for one-stage re-implantation includes removal of the prosthesis and all cement with thorough debridement of bone and soft tissues. A new prosthesis may be implanted in the same procedure. The two-stage procedure includes removal of the prosthesis and all cement with thorough debridement of bone and soft tissues. This may be followed by six or more weeks of antibiotic therapy and then re-implantation of a new prosthesis in a second surgical procedure. A temporary spacer containing antibiotics or other bio-active or radiation substances may be implanted to fill the affected area during the interim between the two surgical procedures (See FIGS. 1-4). The temporary spacer has two roles; it may provide stability for the joint region after removal of the replacement and, by releasing bio-active substances, such as antibiotics, the spacer may protect the joint region from infection resulting at least partially due to colonization by bacteria.

Even though systemic antibiotic therapy may usually be applied after prosthetic infection surgical treatments, it may be unable to reach the infection site in sufficient concentrations to eradicate bacteria. Delivering antibiotics locally with the use of custom made device (total joint spacer or intramedullary nail coating) may eradicate or reduce the infection and the risk of recolonization by providing a very high localized concentration of a bio-active substance. Orthopedic bone cements (PMMA-based polymers with BaSO₄) are currently used to deliver antibiotics to the affected site.

The release kinetics of the bio-active or radiation-emitting substance from the material may be important for the potential clinical benefits to cure and/or prevent sepsis and/or bone infections. The greatest benefits may result from bio-active or radiation-emitting substances released in high concentrations and/or sustained for a length of time targeted at the site of the infection or tumor. High concentrations provide better reduction or eradication of the infection or tumor, while keeping the application localized reduces potential side effects elsewhere in the body.

Another important feature of the coating or materials for orthopedic spacers may be a good level of biocompatibility. Silicone-based elastomers have been shown to be highly biocompatible, such that the use of this material as a carrier or place holder for antibiotics or local radiation sources may not cause added reactions from the surrounding tissue.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 illustrates a healthy femur and femoral head of a human being.

FIG. 2 illustrates a hip joint after a total hip replacement.

FIG. 3 illustrates a deep infection following a total hip replacement.

FIG. 4 illustrates an embodiment of the present invention showing a hip spacer in place to provide localized antibiotic release and temporary stability.

FIG. 5 illustrates an embodiment of the present invention showing improved surface contact with elastomer material.

FIG. 6 is a scanning electron microscopy image of VK100.

FIG. 7 is an X-ray diffraction of VK100.

FIG. 8 is a V-VK100 and test discs on agar plates.

FIG. 9 is 100 dilutions of V-VK100 (left) and VK100 (right).

FIG. 10 is the scheme of six well plate.

FIG. 11 is viable MSC examined with confocal microscopy.

FIG. 12 is viable cells adhered to VK100 surface with ×1000 magnification (a) and ×5000 magnification of a single adhered cell (b).

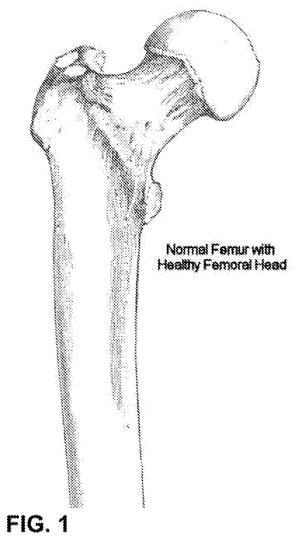

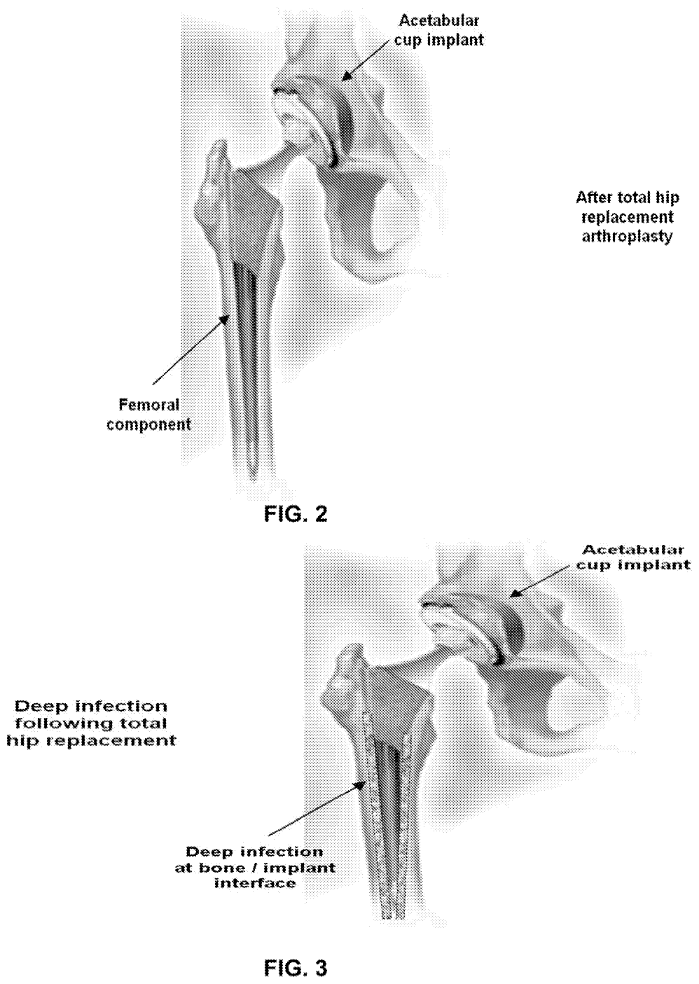

### DESCRIPTION

The following description is merely exemplary in nature and is in no way intended to limit the present disclosure, application, or uses. Throughout this specification, like reference numerals will be used to refer to like elements.

A novel material to create a temporary orthopedic device to use as plug or spacer having a bio-active or radiation-emitting substance after removing an implant in an infected area of the body is provided. Typically, the implant being replaced may be a total hip, knee or shoulder. However, the invention may also be useful in treatment of infected intramedullary nails, compression hip screws and/or other trauma or tumor implants. Additionally, the invention may be useful for treatment of cancerous and benign tumors and lesions; augmentation of screws, plates and pins, encapsulation of implants and foreign bodies, coatings for implants including cardio, neurosurgical, orthopedic and/or spinal implants, and creation of implants for non-load and/or load bearing indications.

Radiation-emitting substances include those designed as radioactive seed implants for internal radiation therapy, which is also known as brachytherapy. They contain radioactive isotopes, which include iodine-125, palladium-103, cesium-131, cesium-127, cobalt-60, iridium-192, ruthenium-106, and radium-226, without limitation.

The spacer may not be expected to replace a load bearing implant. The spacer may only provide some stability for the patient while the infected area may be treated with high localized concentrations of antibiotics. The spacer implant may also include a reinforcement stem or insert to provide added strength. Typically, the indications for this application would include a maximum implantation life in the range of six months (180 days) to two years (730 days).

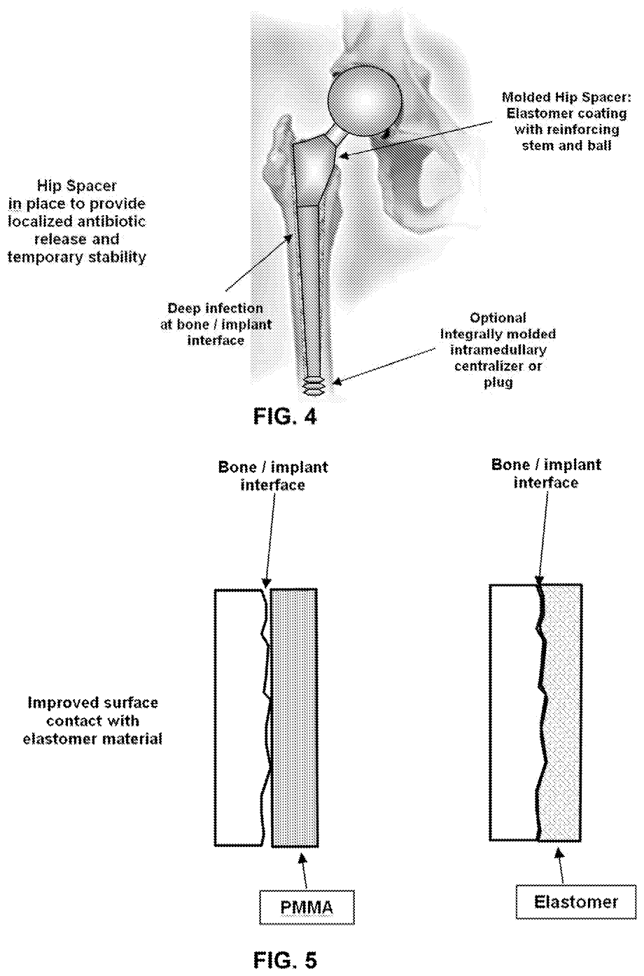

An important advantage of using antibiotic elastomer as the casting or coating material to create the spacer may be the flexibility of the material. There are several reasons why this may be advantageous. For example, the resilience of the elastomer material may be particularly beneficial because it allows better bony apposition, providing larger surface contact with the affected area or tissue (FIG. 5). Bone cement (PMMA) is known to be extremely hard (harder than bone) and may not conform to the surrounding geometry or may be non-compliant with respect to body biomechanics. Often this must be remedied by using additional bone cement applied in a paste to the bone to fill in the gaps. Such use of bone cement in this manner may make the future removal of the spacer very difficult. Since the spacer may only be intended for temporary treatment of an affected area, ease of removal may be important not only to preserve tissue, but also to reduce the time required for the subsequent procedure. Reduced surgical procedure time may be better for patient health and may reduce possible exposure to other pathogens.

Further, making a spacer with an elastomer may allow the use of a larger reinforcing insert, since an elastomer may not be as brittle in thin layers (like PMMA). Therefore, the thickness of the antibiotic containing layer could be determined more by the thickness that may be needed to release the correct dosage of the antibiotic, not limited by a minimum thickness to address the brittle nature of the PMMA or other bone cement material. An additional benefit of using a thinner layer of elastomer on the reinforcing insert may be a stronger implant, since the reinforcing insert may be larger than a reinforcing insert used with PMMA. The volume of elastomer material that may be used to create the temporary spacers may typically range from 25 ml to 300 ml. Spacers produced using PMMA bone cement typically require between 80-400 ml for hip, depending on the size, and 150-200 ml for knee spacers.

Additionally, the use of a compliant material such as the elastomer may allow the user to mold an implantable spacer which incorporates geometry to improve positioning or stability, such as an integral stem centralizer plug. Non-limiting examples of useful geometries include integral rings, elevated pads, ridges, fins, cones, pyramid shapes, and dome shapes.

Further, the use of a compliant material such as the elastomer may have the benefit of providing a cushioning effect where it may be used. The implant may be cushioned by the elastomer.

Another feature of the silicone elastomer use for this invention is the ability of the silicone to gradually release medicinal, bio-active or pharmaceutical or radiation substances. As noted above, the spacer may not be expected to replace a load bearing implant, only to provide some stability for the patient while the infected area may be treated with high localized concentrations of antibiotics. The patient may need the spacer because an infection necessitated the removal of the primary implant. The temporary implantable spacer provides some stability for the patient while the infected area may be treated with high localized concentrations of antibiotics. The silicone elastomer exhibits excellent biocompatibility with surrounding living tissue, both soft tissue and bone. Various antibiotics may be used with the elastomeric spacer including: Vancomycin, Tobramycin and Gentamicin and the like, among others. Any medicament may be used that may provide a benefit to the patient in treating a disease, infection, tumor, and/or injury. For example, chemotherapy, anti-tumor and/or hormonal drugs may also be used when indicated. Moreover, drugs for treating osteoporosis and other bone diseases as well as bone-forming drugs and/or materials may also be used.

In an embodiment of the invention, the antibiotic is added while the silicone elastomer is in a liquid state. The silicone elastomer may be impregnated with antibiotic to a desired concentration for the effective elution of antibiotic in the region of the implant. In an alternative embodiment of the invention, an implant with a cured, solid silicone elastomer coating may be immersed in an antibiotic. This could be done in a chamber using appropriate vacuum. In another embodiment, a pre-formed silicone coated implant may be impregnated with antibiotic prior to and/or during surgery. All known methods of adding an antibiotic, drug, medicament, radiation-emitting substance (also referred to herein as a “radiation seed”), and the like are considered within the scope of this invention.

Another benefit of the silicone elastomer coating on an implant may be the lack of generation or release of significant heat upon solidifying or curing; i.e., the curing of the silicone elastomer may not be considered exothermic. This is in direct contrast to bone cement or PMMA, which is well-known to exhibit extremely large temperature increases during the exothermic polymerization reaction. Also, the elastomer also does not contain toxic chemicals, such as the monomer used in PMMA. A material like a silicone elastomer, which is neither toxic nor exothermic, may not have the same detrimental impact on the antibiotic and/or bio-active substances used. This feature may allow the use of smaller quantities of bio-active substances with the elastomer, because the bio-active substances are not made less effective or less viable due to the extremely harsh environment of PMMA. Additionally, use of the elastomer material may allow creation of spacers utilizing a larger range or spectrum of bio-active substances.

Another aspect of infections relates to biofilms. Formation of a biofilm begins with the attachment of free-floating microorganisms to a surface. Biofilms have been found to be involved in a wide variety of microbial infections in the body, by one estimate 80% of all infections. Infectious processes in which biofilms have been implicated include common problems such as catheter infections, formation of dental plaque, coating contact lenses, and less common but more lethal processes such as infections of permanent implant devices such as joint prostheses and heart valves and neurosurgical shunts. More recently, it has been noted that bacterial biofilms may impair cutaneous wound healing and reduce topical antibacterial efficiency in healing or treating infected skin wounds.

In an embodiment of the present invention, the silicone elastomer may be used to inhibit growth, attachment and/or proliferation of biofilms. A mechanism by which this may be accomplished is that a typically smooth surface of a silicone elastomer may present a difficult surface for the attachment of biofilms. As a result, the growth of biofilms may be inhibited by using silicone elastomers in applications as disclosed herein, for example, coatings on implants and wound-healing applications. Also, applications of silicone on a surface may break the continuity of a biofilm formed thereon which may disrupt the proliferation and growth of the biofilm.

In an embodiment, the composition of the silicone elastomer may comprise two components. For example, component A may comprise:
from 50% to 75% by weight of blended polysiloxanes;
from 10% to 30% by weight of amorphous silica; and
greater than 0.001% by weight of a catalyst such as a platinum catalyst; and
component B may comprise:
   from 50% to 75% by weight of blended polysiloxanes;
   from 10% to 30% by weight of amorphous silica; and
   from 0.5% to 5% by weight of cross-linking agent.

While one embodiment of the composition is described above, the invention is not limited to this embodiment and the scope of the invention is intended to cover all elastomeric polymers and the like.

In an embodiment of the invention, the composition may include an opacifying agent or radiopaque additive. For example, the opacifying agent or radiopaque additive may be selected from one or more of the group consisting of: silver powder, barium sulfate, bismuth trioxide, zirconium dioxide, tantalum powder, tantalum fiber, titanium powder, titanium fiber, calcium sulfate, calcium phosphate, hydroxyapatite, tri-calcium phosphate, gadolinium, iodine, and/or other combinations thereof, as well as future compositions as approved for human and/or animal use.

In an embodiment of the invention, the composition may include a color agent or additive. Although certain elastomeric polymers are white in their cured state, the color may be altered with an appropriate color additive. For example, an elastomeric polymer may be colored so that it may be easily identifiable in the body, especially if the color is not normally found in the body. Revision or removal of the implant may be easier and more complete when all of the colored polymer may be easily identified for removal.

Also, an additional advantage of the quick curing nature of the polymer coupled with its adhesion properties is that it can be used as a wear debris remover. For example, during a revision or removal of a joint, the surgeon may first remove the implant, either the original joint, the primary implant or even the temporary implant from the patient. Then, after the implant and/or joint is removed, the surgeon may add polymer to the removal site cavity to encompass any wear debris in the site, such as bits of PMMA or other wear debris. The polymer cures quickly forming a solid shape that conforms to the site that may then be removed and the encapsulated wear debris may be removed with the polymer.

A further advantage exists in that the polymer may readily conform to irregular anatomy when in liquid form. When cured, the solid polymer may completely fill and fit the volume in which the polymer was inserted. This property of the polymer may be advantageous for several reasons. For example, the complete and shape-conforming fill may provide a better fitting implant and/or improved biomechanical stability. The wear debris removal aspect disclosed above may also be enhanced by the property of the polymer.

The invention may include surface treatments on the reinforcing stem that enhance adhesion of an elastomer onto the reinforcing stem. For example, the reinforcing stem may typically be made of surgical stainless steel. Thus, the surface of the reinforcing stem may be abraded or made rough to enhance adhesion of the polymer.

It should be understood that various changes and modifications to the presently preferred embodiments described herein may be apparent to those having ordinary skill in the art. Such changes and modifications may be made without departing from the spirit and scope of the present invention and without diminishing its attendant advantages.

In non-limiting fashion, the following embodiments are described:

1. A method for forming an implantable spacer device to treat an infected or injured bony structure affected by disease or tumor in a living creature, in particular, a human being, the method comprising:
forming an elastomeric polymer into a spacer, wherein the spacer has a shape sized and configured to fit into a joint;
adding an antibiotic(s), other bio-active substance, or a radiation emitting substance to the elastomeric polymer; and implanting the spacer into a joint.

2. The method of embodiment 1 wherein the spacer is formed using a mold.

3. The method of embodiment 1 wherein the spacer is formed using a mold having a composition selected from the list consisting of: polypropylene, polyethylene, polystyrene, polycarbonate and metal.

4. The method of embodiment 1 wherein the spacer is formed using a plastic mold wherein the plastic mold is disposable and incorporates scored or perforated areas to facilitate removal of the mold from the elastomeric polymer.

5. The method of embodiment 1 wherein the spacer is formed using a plastic mold wherein the plastic mold incorporates scored or perforated areas to facilitate removal of the mold from the elastomeric polymer.

6. The method of embodiment 1 wherein the spacer is formed using a mold wherein the mold is reusable and may be re-sterilized.

7. The method of embodiment 1 wherein the spacer is formed using a plastic mold wherein the plastic mold is translucent so that the user may see whether the mold has filled properly.

8. The method of embodiment 1 wherein the spacer is formed over a reinforcing structure, such as a hip stem.

9. The method of embodiment 1 wherein a spacer is overmolded onto a reinforcing structure which has been surface treated to enhance bonding of the elastomeric polymer to the reinforcing structure.

10. The method of embodiment 1 wherein the spacer is formed using a metal mold wherein the metal mold is reusable and may be re-sterilized.

11. The method of embodiment 1 wherein the antibiotic is selected from Vancomycin, Tobramycin, and Gentamicin.

12. The method of embodiment 1 further comprising the step of adding a tumor treatment to the spacer.

13. The method of embodiment 1 further comprising the step of adding an osteo-inductive substance to the spacer.

14. The method of embodiment 1 further comprising the step of adding a chemotherapy substance to the spacer.

15. The method of embodiment 1 wherein the spacer device is implanted into an orthopedic joint.

16. The method of embodiment 1 wherein the joint is a hip joint.

17. The method of embodiment 1 wherein the joint is a knee joint.

18. The method of embodiment 1 wherein the joint is an elbow joint.

19. The method of embodiment 1 wherein the joint is an ankle joint.

20. The method of embodiment 1 wherein the joint is a wrist joint.

21. The method of embodiment 1 wherein the spacer device is implanted into a spine.

22. The method of embodiment 1 wherein the spacer device is implanted between a pair of adjacent vertebrae.

23. The method of embodiment 1 wherein the elastomeric polymer having the antibiotic(s) and/or other bio-active substance to treat infected or injured bony or soft tissue structures conforms to the bony or soft tissue structures being treated.

24. The method of embodiment 1 further comprising the step of providing a surface treatment onto a reinforcing stem to enhance adhesion of the elastomer onto the reinforcing stem.

25. The method of embodiment 1 wherein the spacer is formed to incorporate an integral stem centralizer plug.

26. The method of embodiment 1 wherein the integral stem centralizer plug has a shape selected from the list of: integral rings, elevated pads, ridges, fins, cone, pyramid and dome shapes.

74. The method of embodiment 1 further comprising the step of providing the spacer with an antibiotic containing layer of elastomeric polymer having a thickness wherein the thickness is chosen to release the correct dosage of the antibiotic.

28. A composition for forming an implantable spacer device to treat an infected or injured bony structure or soft tissue area affected by disease (such as cancer, osteomyelitis, and arthritis), trauma or tumor in a living creature, in particular, a human being, the composition comprising:
an elastomeric polymer; and
an antibiotic(s), other bio-active substance, or a radiation emitting substance.

29. The composition of embodiment 28 wherein the elastomeric polymer comprises polydimethylsiloxane.

30. The composition of embodiment 28 wherein the elastomeric polymer has a Shore A durometer in a range of 10-150.

31. The composition of embodiment 28 further comprising a radiopaque additive.

32. The composition of embodiment 31 wherein the radiopaque additive is selected from one or more of the group consisting of silver powder, barium sulfate, bismuth trioxide, zirconium dioxide, tantalum powder, tantalum fiber, titanium powder, titanium fiber, calcium sulfate, calcium phosphate, hydroxyapatite, tri-calcium phosphate, gadolinium, iodine, and combinations thereof.

33. The composition of embodiment 28 wherein the elastomeric polymer further comprises:
component A having:
   from 50% to 75% by weight of blended polysiloxanes;
   from 10% to 30% by weight of amorphous silica; and
   greater than 0.001% by weight of a catalyst, for example a platinum catalyst; and
component B having:
   from 50% to 75% by weight of blended polysiloxanes;
   from 10% to 30% by weight of amorphous silica; and
   from 0.5% to 5% by weight of cross-linking agent.

34. A method for forming an implantable spacer device to treat an infected or injured bony structure or soft tissue area affected by disease (such as cancer, osteomyelitis, and arthritis), trauma or tumor in a living creature, in particular, a human being, the method comprising:
forming an elastomeric polymer into a spacer, wherein the spacer has a shape sized and configured to fit into a joint; and adding an antibiotic(s),other bio-active substance, or a radiation emitting substance to the elastomeric polymer

35. The method of embodiment 34 further comprising:

forming the spacer is formed over a reinforcing structure.

36. The method of embodiment 35 further comprising: overmolding the spacer onto a reinforcing structure which has been surface treated to enhance bonding of the elastomeric polymer to the reinforcing structure.

37. The method of embodiment 34 further comprising:
providing an integral stem centralizer plug wherein the spacer is formed to incorporate the integral stem centralizer plug.

38. The apparatus of embodiment 37 wherein the integral stem centralizer plug has a shape selected from the list of: integral rings, elevated pads, ridges, fins, cone, pyramid and dome shapes.

39. A method of removing debris from a surgical site, the method comprising the steps of:
applying a elastomeric polymer in a liquid state to the surgical site wherein the elastomeric polymer encompasses the debris and conforms to the surgical site;
allowing the elastomeric polymer to harden in the surgical site; and removing the elastomeric polymer from the surgical site wherein the debris is removed with the elastomeric polymer.

40. A method of restricting growth of biofilm on a surface, the method comprising the step of applying a silicone elastomer to the surface wherein the silicone elastomer forms a smooth coating on the surface.

41. A method of eluting an antibiotic from a material to an affected area, the method comprising the steps of:
adding a bio-active or radiation emitting substance and/or an antibiotic to an elastomeric polymer; and
applying the elastomeric polymer to the affected area.

42. The method of embodiment 41 wherein the step of adding the bio-active substance and/or the antibiotic is performed with the elastomeric polymer in a liquid state.

43. The method of embodiment 41 wherein the step of applying the elastomeric polymer to the affected area is performed with the elastomeric polymer in a liquid state.

44. The method of embodiment 34 further comprising:
forming a washer or spacer from the drug-eluting silicone substance; and using the washer or spacer with a pin, screw, plate or wire in a trauma or restorative surgery.

45. The method of embodiment 34 further comprising:
forming a coating from the drug-eluting in situ curing silicone substance; and using the coating with a plate, IM nail, pin, screw, plate or wire in a trauma or restorative surgery.

46. The method of embodiment 34 further comprising:
forming a coating or layer from the drug-eluting in situ curing silicone substance; and
using the coating to coat, encapsulate, revitalize, repair, or reinitiate bioactivity for a shunt, stent, cage, pacemaker, pacemaker lead, electrode, plate, IM nail, pin, screw, plate or wire in a surgical or outpatient procedure.

47. The method of embodiment 34 further comprising: injecting or placing a coating or mass of the in situ curing elastomer system in the affected area; and encapsulating foreign bodies or undesired structures in the body wherein the encapsulation may be performed with or without drug-elution capability of the system.

48. The method of embodiment 34 further comprising:
placing a coating or mass of the in situ curing elastomer system in the affected area; and
encapsulating foreign bodies or undesired structures in the body wherein the encapsulation may be performed with or without drug-elution capability of the system.

49. The method of embodiment 34 further comprising: forming a mold in the operating room or procedure suite from computer generated 3-D images or other source which accurately provides the geometry required to create the implant specific to the patient and the site.

50. The method of embodiment 34 further comprising:
forming a mold or device in the operating room or procedure suite utilizing 3-D printer technology.

Further non-limiting disclosure is provided in the Examples that follow.

### EXAMPLES

VK100 is a novel silicone based material used in elastoplasty to prevent vertebral fractures. In this study, we assessed the material characterization, antimicrobiological action and biofilm inhibition potential of VK100 and its effect on mesenchymal cells.

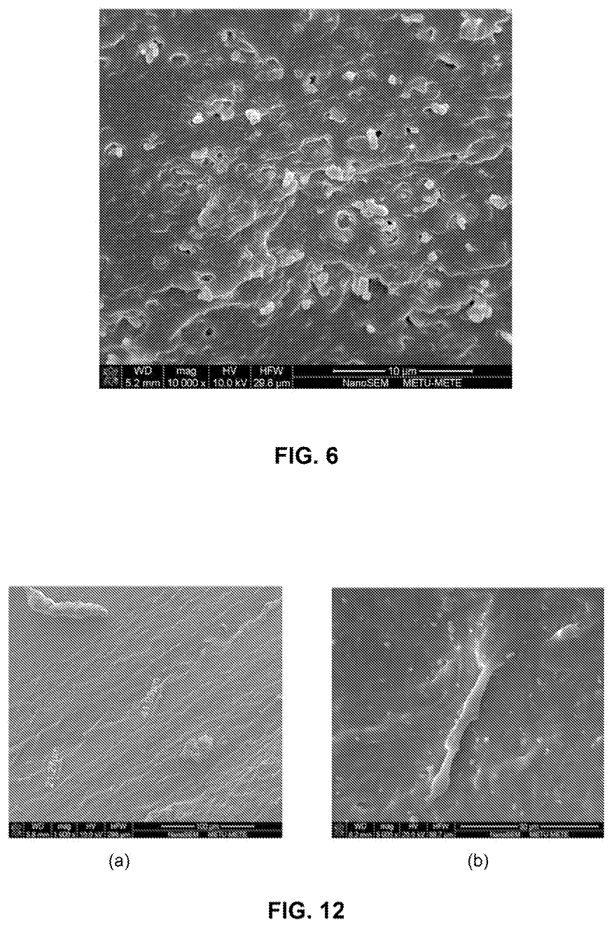

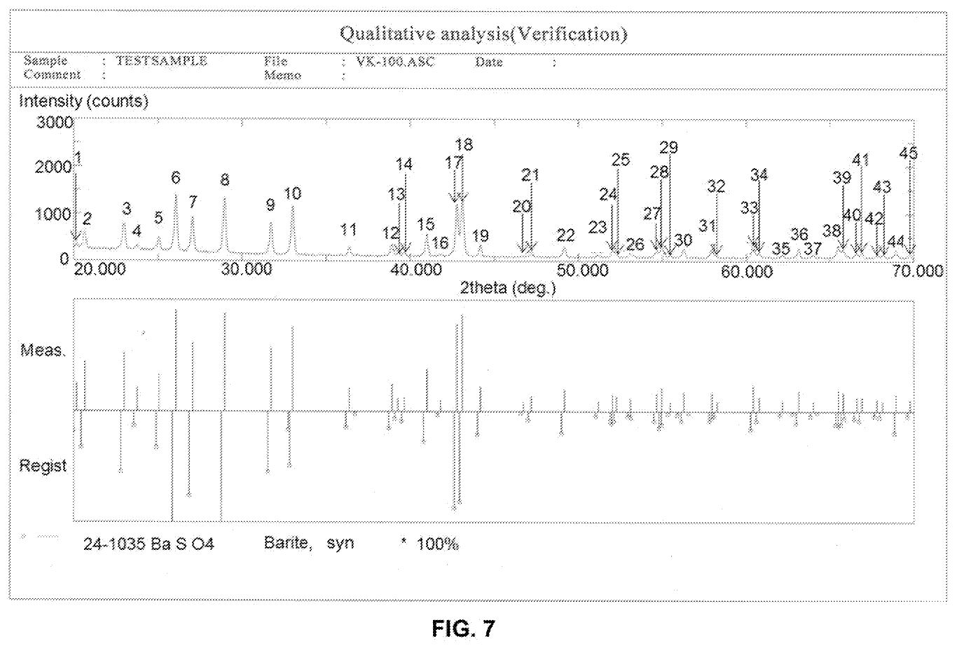

VK 100 was provided by Bonwrx, Ltd. (Michigan, USA). Material was designed as liquid polymer and had its own injection apparatus. To obtain vancomycin containing VK100 (V-VK100), 4.5 g vancomycin powder was spread into 20 ml sterile injector and VK100 was injected. They were mixed with a sterile stick and dried at room temperature for a day. After VK100 was hardened, injector was ceased and cylindrical material was cut into 1.5 cm thick tablets. To obtain VK100 tablets, only VK100 was injected into sterile injector. Surface topography of VK100 was examined by JSM 6400 (Jeol Ltd, Akishima, Japan) scanning electron microscopy found in Middle East Technical University Metallurgical and Materials Engineering Department and found that material surface had a porous structure (FIG. 6). At the same department material quantitative density properties was determined with X-ray diffractometer (XRD) Philips PW/1050 (Philips, Amsterdam, Holland) and found that material structure was similar with BaSO₄ (FIG. 7).

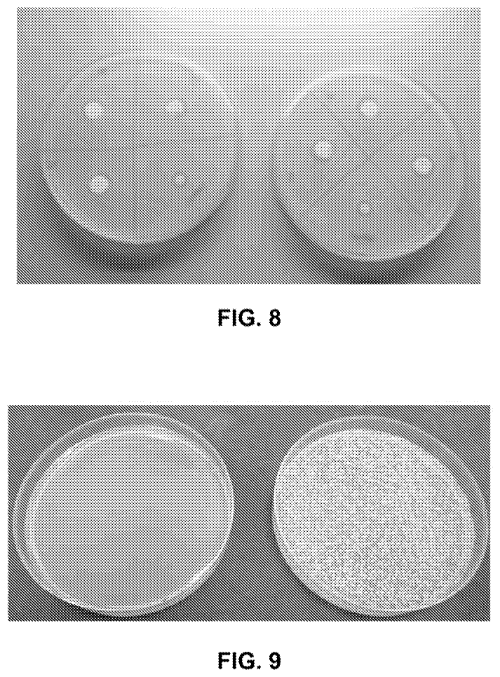

After material characterization, V-VK100 and VK100 tablets were sterilized in Gammapak Sterilization (Gammapak, Tekirdag, Turkey) at 25 kGy for antibiotic susceptibility test, biofilm formation analysis and cell culture studies. For antibiotic susceptibility test, BD BLL Sensi-Disc Antimicrobial Susceptibility Test Discs (Becton Dickinson, New Jersey, USA) were used as control and aseptic conditions were provided during the analysis. Methicillin resistant Staphylococcus aureus (MRSA) (strain, ATCC 2592) was spread onto agar plate (10⁶ cfu/cm³). Test discs and V-VK100 tablets were placed onto agar plates and incubated at 37° C. for 2 days (FIG. 8).

After incubation, inhibition zone diameters were measured. Diameter equal or less than 9 mm were defined as resistant to MRSA, while diameter between 10 and 11 mm were determined as less sensitive. Diameters equal or more than 12 mm were defined as sensitive to MRSA. The results were given below:

| Samples | Day 1 | Day 2 |
|---|---|---|
| V-VK100-sample 1 | 20 mm | 20 mm |
| V-VK100-sample 2 | 18 mm | 18 mm |
| V-VK100-sample 3 | 19 mm | 19 mm |
| V-VK100-sample 4 | 17 mm | 17 mm |
| V-VK100-sample 5 | 16 mm | 16 mm |
| V-VK100-sample 6 | 17 mm | 17 mm |
| Control 1 | 13 mm | 13 mm |
| Control 2 | 13 mm | 13 mm |

As a result, all V-VK100 tablets were sensitive to MRSA.

For biofilm formation analysis, 3 VK100 and 3 V-VK100 tablets were used. In every group, 2 tablets were used for biofilm formation and 1 tablet was used as control. At the first stage of the analysis, tablet surface areas were calculated (Table 1).

TABLE 1
Tablets radius, diameters and surface areas.

| | | | Surface area |
|---|---|---|---|
| Material | Radius (cm) | Diameter (cm) | (cm²) |
| VK100-1 | 1.7 | 0.85 | 2.27 |
| VK100-2 | 1.9 | 0.95 | 2.84 |
| VK100 (control) | 1.9 | 0.95 | 2.84 |
| V-VK100-1 | 1.3 | 0.65 | 1.33 |
| V-VK100-2 | 1.3 | 0.65 | 1.33 |
| V-VK100 (control) | 1.3 | 0.65 | 1.33 |

At second stage of analysis, V-VK100 tablets were incubated with serial dilutions of 10⁻⁵ diluted MRSA culture and this procedure was repeated 2 times during the analysis. At first no colony was observed but at second repeat 4.75×10³ cfu/cm² bacteria were observed. On the other hand, in VK100 total 62×10³ cfu/cm² bacteria were observed. As a result V-VK100 biofilm inhibition potential was % 96 greater than VK100 (FIG. 9.).

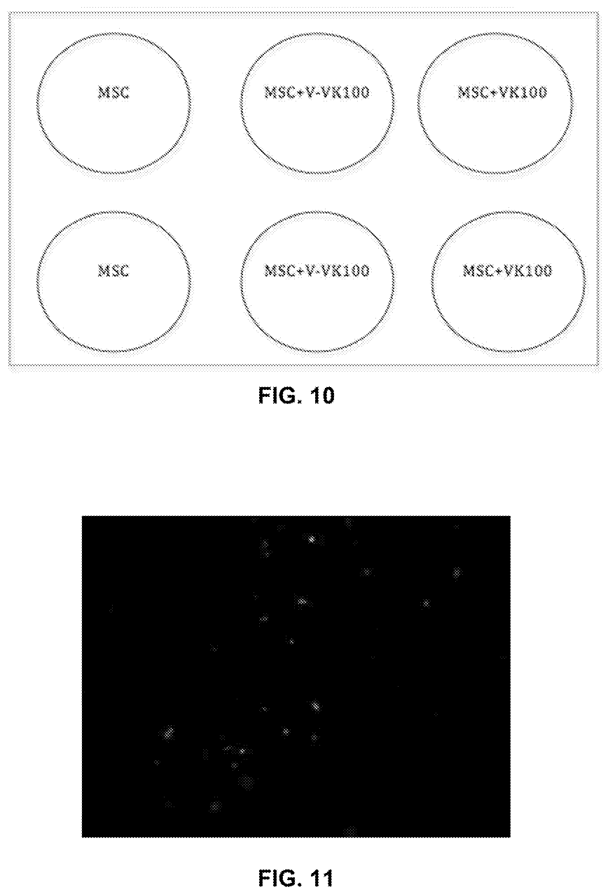

For cell culture studies total 324000 mesenchymal (MSC) cells were used. In a six well plate, two wells contained only MSC cells as control while two wells contained MSC and vancomycin containing VK100 tablet (V-VK100) and another two wells contained MSC and VK100 tablet (FIG. 10).

The plate was incubated once for 3 days and upper row was trypsinized. 4 days later lower row was trypsinized also for seven days cell culture. Later, binding buffer was prepared. For the buffer; 0.2383 g HEPES, 0.8182 g NaCl and 0.0368 g CaCl₂(H₂O)₂ were dissolved in 100 ml double distilled water. Cells were washed with cold phosphate buffer saline (PBS) and centrifuged for 5 minutes at 1500 rpm. Supernatant was poured and 120 ∥l binding buffer was added to cells. 20 μl of cell containing buffer solution was mixed with 20 μl tryphan blue and cells were counted. Cell numbers were found as follows:

| | Day 3 | Day 7 |
|---|---|---|
| MSC | 18000 | 37200 |
| V-VK100 + MSC | 19200 | 32400 |
| VK100 + MSC | 46800 | 26400 |

5 μl Annexin V (Biosource, California, USA) and 10 μl propidium iodide (Invitrogen, California, USA) were added to remaining 100 μl binding buffer and incubated at room temperature for 15 minutes. After that, 4000 μl annexin binding buffer (Biosource, California, USA) was added and cells were analysed in flow cytometer (BD FACS Aria, New Jersey, USA) for apoptosis and necrosis. The percent of cells were given below:

| | Day 3 | Day7 |
|---|---|---|
| | MSC | MSC |
| Late apoptosis | %7.6 | %1.2 |
| Viability | %88.7 | %89.5 |
| Subjected to apoptosis | %2.8 | %2.5 |
| Necrosis | — | %6.7 |
| | MSC + V-VK100 | MSC + V-VK100 |
| Late apoptosis | %18.2 | %2.0 |
| Viability | %70.4 | %79.4 |
| Subjected to apoptosis | %8.9 | %3.9 |
| Necrosis | — | %14.7 |
| | MSC + VK100 | MSC + VK100 |
| Late apoptosis | %7.1 | %7.1 |
| Viability | %84.9 | %65.6 |
| Subjected to apoptosis | %5.5 | %3.1 |
| Necrosis | — | %20.9 |

For confocal laser scanning microscopy 10000 MSC cells were used. Cells were incubated with 50 μl DMEM-LG (Gibco, Invitrogen, California, USA) and 1 μl diI stain (Invitrogen, California, USA) at 37° C. for 30 minutes. After that, 500 μl DMEM-LG was added and centrifugation was done for 5 minutes at 1500 rpm. Supernatant was poured and this procedure was repeated two times. Later, 50 μl cell+DMF 10 solution was poured onto VK100 and incubated at 37° C. for 1 hour. To fix cells, VK100 was washed firstly with PBS and later with paraformaldehyde (PFA). After 30 minutes, again washed with PBS and examined with computer attached Zeiss Axiovert 200M (Zeiss, Oberkochen, Germany) confocal microscopy to see viable cells with red fluorescent (FIG. 11).

After confocal microscopy, same tablet was waited in alcohol gradient (50-100%) for 3 hours and examined with Fei Nova Nano Scanning Electron Microscopy (Fei Company, Oregon, USA) found in Middle East Technical University Metallurgical and Materials Engineering Department to see the cells adhered to VK100 surface (FIG. 12).

For vancomycin release studies, 10 mg and 25 mg vancomycin doses were selected. For each selected dose 6 samples were formed. 6 wells of a 24 well cell culture plate were used to obtain same size samples. Firstly, VK100 was injected and then vancomycin was spread on VK100. After that, the well was mixed with a sterile needle and dried at room temperature for a day. Later, each sample was placed into a flask full with 50 ml PBS. At each measurement time (1, 2, 4, 8, 12, 24, 48, 120 hours and 1, 2, 3, 4, 5, 6 weeks), 1 ml PBS were taken from the flask and replaced with fresh 1 ml PBS. Taken 1 ml PBS were measured in an UV spectrophotometer (Agilent 8453, Agilent Technologies, California, United States) at 280 nm. At the same time 8 solutions (1, 5, 10, 25, 50, 75, 100, 150 μg/ml) were prepared with PBS and vancomycin and measured at 280 nm for calibration curve and validation of the results. The results of the release study will be obtained as soon as possible.

The description herein is merely exemplary in nature and, thus, variations that do not depart from the gist of that which is described are intended to be within the scope of the disclosure. Such variations are not to be regarded as a departure from the spirit and scope of the disclosure.

1. A method for forming an implantable spacer device to treat an infected or injured bony structure, the method comprising:
forming an elastomeric polymer into a spacer, wherein the spacer has a shape sized and configured to fit into a joint; and
adding to the elastomeric polymer at least one of an antibiotic, a bio-active emitting substance, or a radiation emitting substance;
wherein the spacer is configured to be implanted into a joint or an infected or a tumor area.

2. (canceled)

3. The method of claim 1 wherein the spacer is formed using a mold having a composition selected from a list comprising: polypropylene, polyethylene, polystyrene, polycarbonate and metal.

4. The method of claim 1 wherein the spacer is formed using a plastic mold wherein the plastic mold is disposable and incorporates scored or perforated areas to facilitate removal of the mold from the elastomeric polymer.

5. The method of claim 1 wherein the spacer is formed using a plastic mold wherein the plastic mold incorporates scored or perforated areas to facilitate removal of the mold from the elastomeric polymer.

6. (canceled)

7. The method of claim 1 wherein the spacer is formed using a mold that is translucent configured to allow observation of whether the mold has filled to a selected amount.

8. The method of claim 1 wherein the spacer is formed over a reinforcing structure.

9. The method of claim 1 wherein a spacer is overmolded onto a reinforcing structure which has been surface treated to enhance bonding of the elastomeric polymer to the reinforcing structure.

10. (canceled)

11. The method of claim 1 wherein the antibiotic is selected from a list comprising: Vancomycin, Tobramycin, and Gentamicin.

12. The method of claim 1 further comprising adding at least one of a tumor treatment, an osteo-inductive substance, or a chemotherapy substance to the spacer.

13-14. (canceled)

15. The method of claim 1 wherein forming the elastomeric polymer into the spacer includes forming the spacer to be implanted into at least one of an orthopedic joint, a hip joint, a knee joint, an elbow joint, an ankle joint, or a wrist joint.

16-20. (canceled)

21. The method of claim 1 wherein device is forming the elastomeric polymer into the spacer includes forming the spacer to be implanted into a spine.

22. (canceled)

23. The method of claim 1 wherein the elastomeric polymer having the antibiotic(s) and/or other bio-active or radiation emitting substance to treat infected or injured bony or soft tissue structures conforms to the bony or soft tissue structures being treated.

24. The method of claim 1 further comprising providing a surface treatment onto a reinforcing stem to enhance adhesion of the elastomer onto the reinforcing stem.

25. The method of claim 1 wherein the spacer is formed to incorporate an integral stem centralizer plug.

26. (canceled)

27. The method of claim 1 further comprising the step of providing the spacer with an antibiotic containing layer of elastomeric polymer having a thickness wherein the thickness is chosen to release the correct dosage of the antibiotic.

28. A composition for forming an implantable spacer device to treat a bony structure or soft tissue area, the composition comprising:
an elastomeric polymer; and
an antibiotic(s) or other bio-active or radiation emitting substance added to the elastomeric polymer.

29. The composition of claim 28 wherein the elastomeric polymer is polydimethylsiloxane.

30. The composition of claim 28 wherein the elastomeric polymer has a Shore A durometer in a range of 10-150.

31. The composition of claim 28 further comprising a radiopaque additive.

32. (canceled)

33. The composition of claim 28 wherein the elastomeric polymer further comprises:
component A having:
from 50% to 75% by weight of blended polysiloxanes;
from 10% to 30% by weight of amorphous silica; and
greater than 0.001% by weight of platinum catalyst; and
component B having:
from 50% to 75% by weight of blended polysiloxanes;
from 10% to 30% by weight of amorphous silica; and
from 0.5% to 5% by weight of cross-linking agent.

34-40. (canceled)

41. A method of eluting an antibiotic or pharmaceutical or radiation emitting substance from a material to an affected area, the method comprising:
adding a bio-active substance and/or an antibiotic to an elastomeric polymer; and
applying the elastomeric polymer to the affected area.

42. The method of claim 41 wherein adding the bio-active substance and/or the antibiotic is performed while the elastomeric polymer in in a liquid state.

43. The method of claim 41 wherein the step of applying the elastomeric polymer to the affected area is performed with the elastomeric polymer in a liquid state.

44. The method of claim 1 further comprising:
forming a washer or spacer from the drug-eluting silicone substance; and
using the washer or spacer with a pin, screw, plate or wire in a trauma or restorative surgery.

45. The method of claim 1 further comprising:
forming a coating from the drug-eluting in situ curing silicone substance;
and using the coating with a plate, intramedullary nail, pin, screw, plate or wire in a trauma or restorative surgery.

46. The method of claim 1 further comprising:
forming a coating or layer from the drug-eluting in situ curing silicone substance; and
using the coating to coat, encapsulate, revitalize, repair, or reinitiate bioactivity for a shunt, stent, cage, pacemaker, pacemaker lead, electrode, plate, IM nail, pin, screw, plate or wire in a surgical or outpatient procedure.

47. The method of claim 1 further comprising:
injecting or placing a coating or mass of the in situ curing elastomer system in the affected area; and
encapsulating foreign bodies or undesired structures in the body wherein the encapsulation may be performed with or without drug-elution capability of the system.

48-50. (canceled)
